(19) United States
(12) Patent Application Publication
(10) Pub. No.: US 2012/0303011 A1
(43) Pub. Date: Nov. 29, 2012

(54) Catheters and Methods for Identification and Treatment of Bodily Passages

(75) Inventor: Darin Schaeffer, Bloomington, IN (US)

(73) Assignee: Cook Medical Technologies LLC, Bloomington, IN (US)

(21) Appl. No.: 13/304,432

(22) Filed: Nov. 25, 2011

Related U.S. Application Data
(60) Provisional application No. 61/417,422, filed on Nov. 27, 2010.
(60) Provisional application No. 61/510,573, filed on Jul. 22, 2011.

(51) Int. Cl. A61B 18/18

(52) U.S. Cl. 606/16

(57) ABSTRACT

Catheters useful in the identification and treatment of bodily passages, such as sinus cavities, are provided. A catheter includes an elongate main body defining an inflation lumen and an optic lumen, an inflatable balloon attached to the distal end of the main body, and a fiber optic disposed in the optic lumen. When connected to a light source, the fiber optic emits light radially along a portion of the balloon and axially from the distal end of the main body. Methods of identifying and treating bodily passages, such as sinus cavities, are also described.

### CROSS-REFERENCE TO RELATED APPLICATIONS

This application claims priority to U.S. Provisional Application No. 61/417,422 filed on Nov. 27, 2010 and U.S. Provisional Application No. 61/510,573 filed on Jul. 22, 2011. The disclosure of each of these related applications is hereby incorporated into this disclosure in its entirety.

### FIELD

The invention relates generally to the field of medical devices. More particularly, the invention relates to catheters useful in the transcutaneous identification and treatment of bodily passages, such as sinus cavities. The invention also relates to methods of identifying and treating bodily passages.

### BACKGROUND

It is sometimes necessary or otherwise desirable to dilate bodily passages and/or cavities, such as sinus cavities, using a balloon catheter. For example, when sinus cavities become blocked, balloon sinuplasty, the dilation of the sinus using a balloon catheter, provides an alternative to radical surgical approaches to unblocking the sinus.

Conventional sinuplasty procedures place a balloon catheter over a previously-placed wireguide and are complicated by the need to use x-ray or other visualization equipment to verify positioning of the balloon and wireguide in the proper sinus cavity prior to the actual dilation procedure.

Lighted wireguides have been developed to facilitate the visualization process, but still present significant drawbacks. For example, while the wireguide can be visualized transcutaneously following placement in the sinus cavity, the light of the wireguide is blocked once the balloon is tracked over the tip of the wireguide, preventing the user from confirming final placement of the actual balloon prior to dilation. Another drawback is that multiple devices must be advanced to a treatment site which increases the complexity of the procedure and the amount of time required to complete the procedure. Furthermore, the lighted wireguide does not convey any information to the user about the balloon or the area of the cavity that will be contacted and/or treated upon its expansion.

A need exists, therefore, for improved catheters and methods of identifying and treating bodily passages.

### SUMMARY

Several exemplary catheters are described herein. The catheters can be used to identify and treat bodily passages, such as sinus cavities.

An exemplary catheter comprises an elongate main body having a proximal end and a distal end and defining an inflation lumen and an optic lumen; a balloon attached to the distal end of the main body and adapted to move between deflated and inflated configurations, the balloon having a proximal portion, a distal portion, and an intermediate portion disposed between the proximal and distal portions, the intermediate portion defining a maximum diameter of the balloon when the balloon is in the inflated configuration; and a fiber optic disposed in the optic lumen and adapted to be operatively connected to a light source, the fiber optic adapted to emit light radially along the intermediate portion of the balloon and axially from the distal end of the main body.

Another exemplary catheter comprises an elongate main body having a proximal end and a distal end, the main body defining an inflation lumen and an optic lumen; a balloon attached to the distal end of the main body and adapted to move between deflated and inflated configurations, the balloon having an opaque proximal portion, an opaque distal portion, and a transparent intermediate portion disposed between the proximal and distal portions, the intermediate portion defining a maximum diameter of the balloon when the balloon is in the inflated configuration; and a fiber optic disposed in the optic lumen and adapted to be operatively connected to a light source, the fiber optic adapted to emit light radially along the intermediate portion of the balloon and axially from the distal end of the main body.

Another exemplary catheter comprises an elongate main body having a proximal end and a distal end and defining an inflation lumen and an optic lumen; a balloon attached to the distal end of the main body and adapted to move between deflated and inflated configurations, the balloon having a proximal portion, a distal portion, and an intermediate portion disposed between the proximal and distal portions, the intermediate portion defining a maximum diameter of the balloon when the balloon is in the inflated configuration; and a fiber optic disposed in the optic lumen and adapted to be operatively connected to a light source, the fiber optic having proximal and distal masked portions and an unmasked portion disposed between the proximal and distal masked portions and adapted to emit light radially along the intermediate portion of the balloon. The fiber optic also adapted to emit light axially from the distal end of the main body.

Methods of identifying and treating bodily passages are also described. An exemplary method comprises advancing a catheter according to an embodiment of the invention into a bodily passage of a patient using conventional interventional techniques. Another step comprises activating the light source, causing radially-directed light to emit from the fiber optic along the intermediate portion of the balloon and axially-directed light to emit from the distal end of the catheter. Another step comprises visualizing the axially-directed light through the skin of the patient. Another step comprises advancing the distal end of the catheter into the portion of the bodily passage intended to be treated by the catheter until the user determines that the intensity of the axially-directed light is indicative of positioning of the distal end of the catheter in the portion of the bodily passage intended to be treated. Another step comprises visualizing the radially-directed light through the skin of the patient. Another step comprises confirming that the radially-directed light is illuminating a portion of the skin of the patient that is located superficially to the portion of the bodily passage intended to be treated by the catheter. Another step comprises inflating the balloon of the catheter. Another step comprises deflating the balloon of the catheter. Another step comprises withdrawing the catheter from the bodily passage.

Another exemplary method comprises advancing a catheter according to an embodiment of the invention into a bodily passage of a patient using conventional interventional techniques. In this method, a dual-balloon catheter in which the second balloon includes one or more regions defining pores is used. Another step comprises activating the light source, causing radially-directed light to emit from the fiber optic along the intermediate portion of the balloon only in the regions on the second balloon that define the pores, and axially-directed light to emit from the distal end of the catheter. Another step comprises visualizing the axially-directed light through the skin of the patient. Another step comprises advancing the distal end of the catheter into the portion of the bodily passage intended to be treated by the catheter until the user determines that the intensity of the axially-directed light is indicative of positioning of the distal end of the catheter in the portion of the bodily passage intended to be treated. Another step comprises visualizing the radially-directed light through the skin of the patient. Another step comprises confirming that the radially-directed light is illuminating a portion of the skin of the patient that is located superficially to the portion of the bodily passage intended to be treated by the catheter. Another step comprises inflating the first balloon of the catheter. Another step comprises introducing fluid into the second inflation lumen with sufficient pressure to force fluid through the pores defined by the second balloon. Another step comprises deflating the first balloon of the catheter. Another step comprises withdrawing the catheter from the bodily passage.

Another exemplary method comprises advancing a catheter according to an embodiment of the invention into a bodily passage of a patient using conventional interventional techniques. Another step comprises activating the light source, causing axially-directed light to emit from the distal end of the catheter. Another step comprises visualizing the axially-directed light through the skin of the patient. Another step comprises advancing the distal end of the catheter into the portion of the bodily passage intended to be treated by the catheter until the user determines that the intensity of the axially-directed light is indicative of positioning of the distal end of the catheter in the portion of the bodily passage intended to be treated. Another step comprises confirming that the axially-directed light is illuminating a portion of the skin of the patient that is located superficially to the portion of the bodily passage intended to be treated by the catheter. Another step comprises inflating the balloon of the catheter. Another step comprises deflating the balloon of the catheter. Another step comprises withdrawing the catheter from the bodily passage.

Additional understanding of the devices and methods contemplated and/or claimed by the inventor can be gained by reviewing the detailed description of exemplary embodiments, presented below, and the referenced drawings.

### BRIEF DESCRIPTION OF THE DRAWINGS

FIG. 1 is a side view of a first exemplary catheter.

FIG. 1A is a sectional view of the catheter illustrated in FIG. 1, taken along line 1A-1A.

FIG. 2 is a side view of a second exemplary catheter.

FIG. 3 is a side view of a third exemplary catheter.

FIG. 3A is a sectional view of the catheter illustrated in FIG. 3, taken along line 3A-3A.

FIG. 3B is a sectional view of the catheter illustrated in FIG. 3, taken along line 3B-3B.

FIG. 3C is a sectional view of the catheter illustrated in FIG. 3, taken along line 3C-3C.

FIG. 4 is a flowchart representation of an exemplary method of treatment.

FIG. 5 is a flowchart representation of another exemplary method of treatment.

### DETAILED DESCRIPTION

The following description of exemplary embodiments provides illustrative examples of that which the inventor regards as his invention. As such, the embodiments discussed herein are merely exemplary in nature and are not intended to limit the scope of the invention, or its protection, in any manner. Rather, the description of these exemplary embodiments serves to enable a person of ordinary skill in the relevant art to practice the invention.

As used herein, the term “bodily passage” refers to any passage within the body of an animal, including, but not limited, to humans, and includes elongate passages, such as blood vessels, and cavities, such as sinus cavities. The term “cavity” refers to an air-filled space within the body. The term “sinus cavity” refers to an air-filled space located within the body and in communication with the nasal cavity.

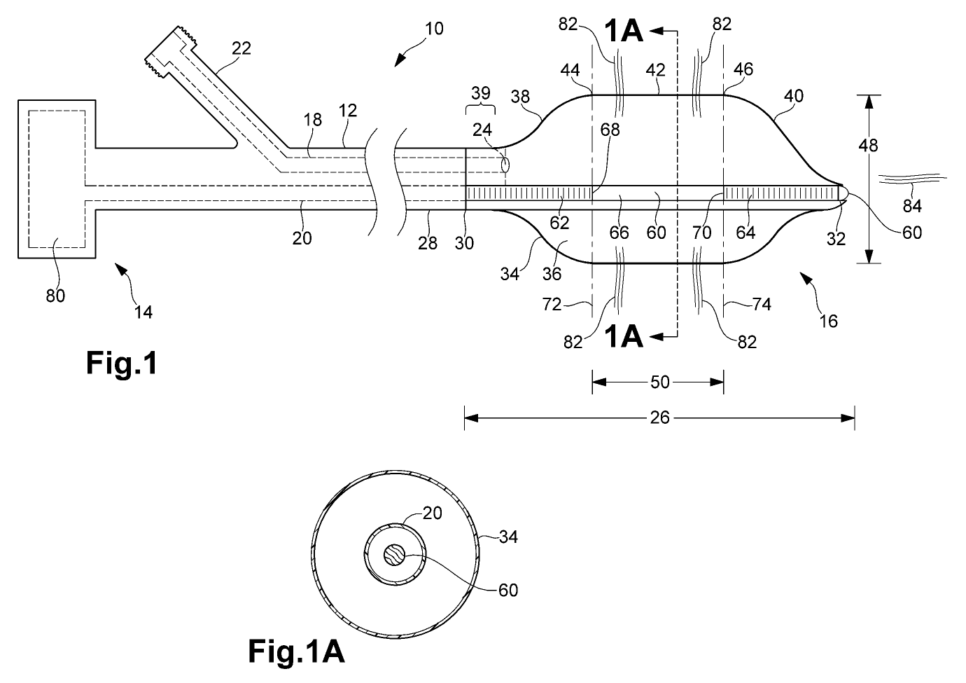

FIGS. 1 and 1A illustrate a first exemplary catheter 10. The catheter 10 includes an elongate main body 12 extending between proximal 14 and distal 16 ends. The main body 12 defines an inflation lumen 18 and an optic lumen 20. The inflation lumen 18 extends between an inflation port 22 and an opening 24 positioned proximal to the distal end 16 of the main body 12. The optic lumen 20 extends from the proximal end 14 to the distal end 16 of the main body 12.

The main body 12 includes a translucent tip 26 and an opaque portion 28. The translucent tip 26 is formed of translucent material or substantially translucent material, while the opaque portion 28 is formed of opaque or substantially opaque material. Thus, the translucent tip 26 is adapted to allow light to pass from within the main body 12, i.e., from within one or both of the lumens 18, 20, to the environment external to the main body 12, while the opaque portion 28 is adapted to block or prevent light from passing from within the main body 12 to the environment external to the main body 12.

Any material having a suitable degree of translucence can be used for the translucent tip 26. Similarly, any material having a suitable degree of opaqueness can be used for the opaque portion 28. The degree of translucence and opaqueness for the translucent tip 26 and the opaque portion 28, respectively, are not critical and a skilled artisan will be able to select suitable degrees and suitable materials for these portions in a particular catheter based on various considerations, including the nature of the body vessel and/or cavity within which the catheter is intended to be used, the expected position of the treatment site relative to the skin of an intended patient group, and other considerations. The only requirement is that the translucent tip 26 is more translucent than the opaque portion 28, and that the opaque portion 28 is more opaque than the translucent tip 26.

The translucent tip 26 and the opaque portion 28 are attached to each other at junction 30. Junction 30 can comprise any suitable attachment between members, and skilled artisans will be able to select an appropriate attachment for a particular catheter based on various considerations, including the nature of the materials used for the translucent tip 26 and the opaque portion 28. Examples of suitable attachments include a butt joint (as illustrated in FIG. 1), a threaded joint, an overlapped joint, and any other suitable attachment between members. An example method of attaching to the translucent tip 26 and the opaque portion 28 includes heating the proximal end of the translucent portion 26 and/or the distal end 28 of the opaque portion and compressing these ends together. In addition, adhesives, connectors, and other suitable structure and/or compositions can be used to form a suitable junction 30. No matter the type or form of attachment used, junction 30 should sufficiently seal the translucent tip 26 and opaque portion 28 to each other so that fluid within the inflation lumen 18 cannot pass through the junction 30 during inflation of the balloon 34, described more fully below. Alternatively, the translucent tip 26 and opaque portion 28 can be integrated components with varying translucence and opaqueness.

A balloon 34 is attached to the distal end 16 of the main body 12 and positioned about the translucent tip 26. The material of the balloon 34 and the portion of the exterior surface of the main body 12 positioned within the balloon 34 (e.g., exterior surface of the distal end of the inflation lumen 18, and a portion of the optic lumen 20) define an inflation chamber 36. The balloon 34 is positioned on the distal end 16 of the main body 12 such that the opening 24 is in communication with the inflation chamber 36. With this structural arrangement, the balloon 34 is adapted to move between deflated and inflated configurations as a fluid is moved into and out of the inflation chamber 36 via the inflation lumen 18 and the opening 24.

A user inflates the balloon 34 by introducing an appropriate fluid, such as saline, into the inflation lumen 18 until the fluid passes through opening 24 and into the inflation chamber 36. The resulting pressure placed on the inner surface of the balloon 34 by the fluid causes the balloon 34 to inflate and adopt the inflated configuration. To move the balloon to the deflated configuration, vacuum pressure can be applied to the inflation lumen 18 to remove fluid located within the inflation chamber 36 via the opening 24, resulting in the balloon 34 collapsing against the distal end 16 of the main body 12. FIGS. 1 and 1A illustrate the balloon 34 in the inflated configuration.

Additional structure can be attached to the catheter 10 to facilitate the inflation and deflation of the balloon 34 as described above. For example, a syringe (not illustrated) or other suitable structure can be attached to the inflation port 22 using any suitable connection, such as a luer lock connection. The fluid can be stored within the syringe and inflation lumen 18, and can be introduced into and removed from the inflation chamber 36 by operating the syringe using conventional practices.

The balloon 34 is positioned over the translucent tip 26 of the main body 12, extending from the junction 30 to the atraumatic tip 32. The balloon 34 has an axial length extending along the longitudinal axis of the main body 12 and has a proximal portion 38, a distal portion 40, and an intermediate portion 42 disposed between the proximal 38 and distal 40 portions. The intermediate portion 42 extends from an intermediate portion proximal end 44 to an intermediate portion distal end 46. As best illustrated in FIG. 1, the intermediate portion 42 defines a maximum diameter 48 of the balloon 34 when the balloon 34 is in the inflated configuration. The maximum diameter 48 is uniform along the axial length of the intermediate portion 42 of the balloon 34. In contrast, the proximal 38 and distal 40 portions of the balloon 34 have diameters that taper along the axial length of the respective portion 38, 40, from a minimal diameter positioned at the locations where the portions 38, 40 contacts the main body 12 to the maximum diameter 48 at the locations where the portions 38, 40 meets the intermediate portion 42.

The minimal diameter of the proximal portion 38 that contacts the main body 12 includes a portion 39 of the balloon 34 that continuously contacts and is attached to the main body 12. Portion 39 can comprise any suitable length. Examples of suitable lengths for portion 39 include, but are not limited to, lengths within the range from about 1 mm to about 5 mm. An exemplary length for portion 39 is about 3 mm. Portion 39 of the balloon can be attached to the main body 12 using any suitable method of attachment, and skilled artisans will be able to select an appropriate method of attachment according to a particular embodiment based on various considerations, including the intended use of the catheter, and other considerations. Examples of suitable methods for attaching the balloon 34 to the main body 12 include using adhesive, heat bonding, and any other method considered suitable. Alternatively, the proximal end of the balloon 34 can be attached to the main body 12 and portion 39 can be omitted.

As best illustrated in FIG. 1, the balloon 34 has a working length 50 that extends along the lengthwise axis of the main body 12 from the intermediate portion proximal end 44 to the intermediate portion distal end 46. The working length 50 is an axial portion of the balloon 34 that has the maximum diameter 48 of the balloon 34 when the balloon 34 is in the inflated configuration. Thus, in use, the working length 50 of the balloon 34 corresponds to the portion of the balloon 34 extending along the axial length of the main body 12 that contacts an inner wall or tissue of a body vessel or cavity when the balloon 34 is in the inflated configuration while disposed within the body vessel or cavity.

A fiber optic 60 is disposed within the optic lumen 20 of the main body 12. The fiber optic 60 includes a proximal masked portion 62, a distal masked portion 64, and an intermediate portion 66 disposed between the proximal masked portion 62 and the distal masked portion 64. The intermediate portion 66 extends from an intermediate portion proximal end 68 to an intermediate portion distal end 70. Each of the proximal masked portion 62 and the distal masked portion 64 is adapted to block or prevent light from escaping the fiber optic 60 in a radial direction with respect to the lengthwise axis of the fiber optic 60. In contrast, the intermediate portion 66 is adapted to allow light to escape from the fiber optic 60 in a radial direction with respect to the lengthwise axis of the fiber optic 60.

The atraumatic tip 32 comprises a soft, clear, rounded end attached to the distal end of the fiber optic 60. The atraumatic tip 32 can be, however, attached to the distal end 16 of the main body 12, or be formed from the same material as, and/or integrated with, the main body 12. While the atraumatic tip 32 has been described as a soft, clear, rounded element, skilled artisans will be able to select a suitable atraumatic tip according to a particular embodiment based on various considerations, including the nature of the body vessel and/or cavity within which the catheter is intended to be used, and other considerations. Examples of suitable atraumatic tip configurations include forming the atraumatic tip of a semi-rigid material with any suitable translucence and having any suitable geometric shape. Examples of suitable methods of attaching the atraumatic tip to the distal end of the fiber optic and/or the distal end 16 of the main body 12 include using heat bonding, threaded components, adhesives, and any other suitable method.

The fiber optic 60 is advantageously fixed in position relative to the main body 12. For example, the distal end of the fiber optic 60, the distal end of the optic lumen 20, the distal end of the balloon 34, and/or the atraumatic tip 32 can be fused together, such as by heat bonding the elements to one another. While the distal end of the fiber optic 60, the distal end of the optic lumen 20, the distal end of the balloon 34, and/or the atraumatic tip 32 have been described as being fused together using a heat bond, skilled artisans will be able to select an appropriate method of attachment according to a particular embodiment based on various considerations, including the nature of the body vessel and/or cavity within which the catheter is intended to be used, and other considerations. Examples of suitable methods of attachment include using adhesive, threaded components, or any other suitable method.

While the fiber optic 60 has been described and illustrated as being disposed within the optic lumen 20 of the main body 12, the fiber optic 60 can be otherwise configured. For example, the optic lumen 20 can terminate parallel to, proximate, or distal to opening 24, and the fiber optic 60 can extend through the chamber 36 and the fiber optic 60 and/or the atraumatic tip 32 can be attached to the distal end of the balloon 34.

If considered necessary or desirable, any suitable structural adaptions can be applied to the intermediate portion 66 of the fiber optic 60 to accomplish the desired radial emission of light, and skilled artisans will be able to select an appropriate structural adaptation for a particular catheter based on various considerations, including the nature of the material used on the fiber optic 60. Examples of suitable structural adaptations include scuffing of the exterior surface of the fiber optic 60, partially or completely removing the cladding of the fiber optic 60 in the intermediate portion 66, and any other suitable structural adaptations. It is noted, though, that a fiber optic without such structural adaptations is expected to provide suitable radial emission of light.

The fiber optic 60 is positioned within the catheter 10 relative to the main body 12 in a manner that facilitates visualization of a portion of a body vessel or cavity, such as a sinus cavity, that will be contacted by the working length 50 of the balloon 34 when the balloon 34 is placed into the expanded configuration within the body vessel or cavity, as described more fully below. As best illustrated in FIG. 1, the structural arrangement of the fiber optic 60 and the main body 12 include several factors that contribute to this desired function of the catheter 10. First, the intermediate portion 66 has the same axial length, or substantially the same axial length, as the working length 50 of the balloon 34. Second, a first orthogonal axis 72 intersects the lengthwise axis of the main body 12 and the intermediate portion proximal ends 44, 68 of the balloon 34 and fiber optic 60. Similarly, a second orthogonal axis 74 intersects the lengthwise axis of the main body 12 and the intermediate portion distal ends 46, 70 of the balloon 34 and fiber optic 60. Third, the proximal masked portion 62 of the fiber optic 60 is positioned under the proximal portion 38 of the balloon, and the distal masked portion 64 of the fiber optic 60 is positioned under the distal 40 portion of the balloon.

With this structural arrangement of the fiber optic 60 and the main body 12, the intermediate portion 66 of the fiber optic 60, which is adapted to emit light from the fiber optic 60 in a radial direction with respect to the lengthwise axis of the fiber optic 60, extends along the same axial length of the lengthwise axis of the main body 12 as the working length 50 of the balloon 34. To preserve this desired structural arrangement, it is considered advantageous to fix the position of the fiber optic 60 relative to the main body 12, such as by fixedly attaching the fiber optic 60 to the main body 12 so that no relative movement between these elements can occur. Any suitable means for and/or techniques can be used to accomplish this fixed positioning between the fiber optic 60 and main body 12, including adhesives, mechanical connectors, and any suitable structure and/or technique for securing the fiber optic 60 within the optic lumen 20. Alternatively, the fiber optic 60 can be slidably and releasably disposed within the optic lumen 20.

A light source 80 is attached to or otherwise associated with the fiber optic 60 at the proximal end 14 of the catheter 10 such that light generated by the light source 80 is able to travel through the light path defined by the fiber optic 60. Because of the structural adaptations in the intermediate portion 66, the fiber optic 60 emits radially-directed light 82 from the intermediate portion 66 of the light source 80 and axially-directed light 84 from the distal end of the fiber optic 60 when the light source is energized and passing light into the light path defined by the fiber optic 60. Furthermore, because of the structural arrangement of the fiber optic 60 and the main body 12, as described above, the radially-directed light 82 emitted from the intermediate portion 66 of the fiber optic 60 passes through the balloon 34 along its working length 50. Thus, the working length 50 allows light emitted from the intermediate portion 66 of the fiber optic 60 to pass through the material of the balloon to an external environment. Alternatively, the light source 80 can be omitted and provided separately from the catheter. When using a catheter according to one of these embodiments, a user attaches or otherwise associates a light source with the fiber optic either before beginning use of the catheter or immediately prior to visualization with the catheter.

It is noted that, while a single fiber optic 60 is described above and illustrated in the Figures, two different fiber optics could be used to independently provide the axially- and radially-directed light. In these embodiments, two fiber optics extend through the same or different optic lumens defined by the catheter and are operatively connected to the same or two different light sources. One or more switches can be provided to allow a user to selectively turn on and off each of the fiber optics, such as by blocking light from entering the fiber optic to be “turned off.” This would allow a user to focus solely on either the axially- or radially-directed light during a procedure.

Using the catheter 10, a care provider is able to verify the placement of the catheter 10 within a bodily passage and/or cavity by activating the light source 80 and locating the axially-directed light 84 emitting from the distal end 16 of the catheter 10. Furthermore, the care provider can identify the portion of a bodily passage and/or cavity that will be contacted by the balloon by activating the light source 80 and locating the radially-directed light 82 emitting from the catheter 10 along the working length 50 of the balloon. Locating the axially-directed and/or radially-directed light can be accomplished transcutaneously, and/or with a scope.

Any suitable materials can be used for the main body 12, balloon 34, fiber optic 60 and other elements of the catheter 12, and skilled artisans will be able to select appropriate materials for a particular catheter based on various considerations, such as the nature of the body vessel and/or cavity within which the catheter is intended to be used. Examples of suitable materials include plastics and other materials used in the manufacture of conventional catheters.

Any suitable fiber optic can be used in the catheter 10. Commercially-available fiber optics considered suitable for use in the catheter 10 include plastic and glass fiber optics, with or without cladding.

Any suitable light source can be used in the catheter, and skilled artisans will be able to select an appropriate light source for a particular catheter based on various considerations, including the location of the body vessel and/or cavity within which the catheter is intended to be used. Commercially-available light sources considered suitable for use in the catheter 10 include xenon, laser, LED, halogen, and other suitable light sources.

While the fiber optic 60 has been described and illustrated as configured to emit radially directed light 82 and axially directed light 84, the main body 12, optic lumen 20, balloon 34, and/or optic fiber 60 can be adapted to emit only axially directed light 82 from the distal end 16 of the main body 12 and the distal portion 40 of the balloon 34. For example, the translucent tip 26 can be omitted and the main body 12 can comprise an opaque portion 28 that extends to distal end 16. In an additional example, the optic lumen 28 can be masked along its length except for the distal end of the optic lumen 28 allowing for only axially-directed light to be emitted from the fiber optic 60. In a further example, the balloon 34, and/or fiber optic 60, can mask the length of the fiber optic 60 allowing for only axially-directed light to be emitted by the distal end of the fiber optic 60. Each example of providing only axially-directed light 84 can be used separately, or in combination with one another.

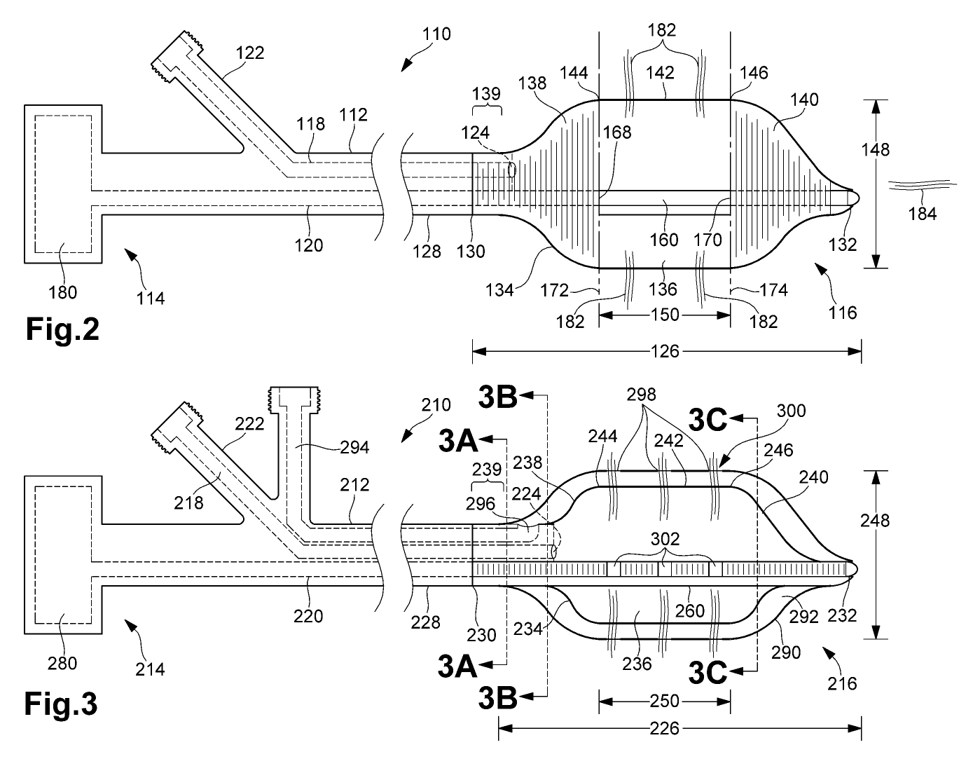

FIGS. 2 and 2A illustrate a second exemplary catheter 110. The catheter 110 illustrated in FIG. 2 is similar to the first exemplary catheter 10 illustrated in FIGS. 1 and 1A, except as detailed below. Thus, the catheter 110 includes an elongate main body 112 extending between proximal 114 and distal 116 ends. The main body 112 defines an inflation lumen 118 and an optic lumen 120. The inflation lumen 118 extends between an inflation port 122 and an opening 124 positioned proximal to the distal end 116 of the main body 112. The optic lumen 120 extends from the proximal end 114 to the distal end 116 of the main body 112.

The catheter 110 includes an alternative structure for limiting radially-directed light 182 to the working length 150 of the balloon 134. In contrast to catheter 10, which includes proximal masked portion 62 and distal masked portion 64 on the fiber optic 60, the balloon 134 in catheter 110 is masked. Specifically, the proximal 138 and distal 140 portions of the balloon 134 are opaque, while the intermediate portion 142 of the balloon 134 remains transparent, allowing light emitted from the fiber optic 60 to pass through the material of the balloon to an external environment.

Any suitable structure and/or process can be used to mask the proximal 138 and distal 140 portions of the balloon 134 and skilled artisans will be able to determine an appropriate structure and/or process for a particular catheter based on various considerations, such as the material used in the formation of the balloon 134. Examples of suitable structure include a balloon formed of an opaque material or materials at the proximal and distal portions and of a translucent or transparent material or materials at the intermediate portion. Alternatively, a conventional transparent balloon can be used and a mask can be applied to the proximal and distal portions, such as by spraying an opaque coating onto these portions.

The use of masked proximal 138 and distal 140 portions on the balloon 134 is considered advantageous at least because it positions the masking nearer to the intermediate portion 142 of the balloon 134 and is expected to result in a more narrow band of radially-directed light 182 emitting from the catheter 110 than the band of light that would be emitted by the catheter 10 described above. By placing the masking nearer to the intermediate portion 142 of the balloon 134, less leakage of light from the intermediate portion 142 is expected to occur. In turn, this is expected to facilitate more accurate identification of the location within body vessels and/or cavities that will be contacted and treated by the catheter 110.

A combination of the first 10 and second 110 exemplary catheters is also contemplated. In this embodiment, the fiber optic 60 of the first catheter 10, illustrated in FIGS. 1 and 1A, is combined with the main body 112 and balloon 134 of the second catheter 110. This combination is expected to be advantageous at least because it provides a structure having dual masking, i.e., masking of radially-directed light emitting from the fiber optic 60 by both the proximal masked portion 62 and distal masked portion 64 of the fiber optic 60, and by the opaque proximal 138 and distal 140 portions of the balloon 134. This combination is expected to further facilitate more accurate identification of the location within body vessels and/or cavities that will be contacted and treated by the catheter by providing a tight alignment of radially-directed light and the working length 150 of the balloon 134.

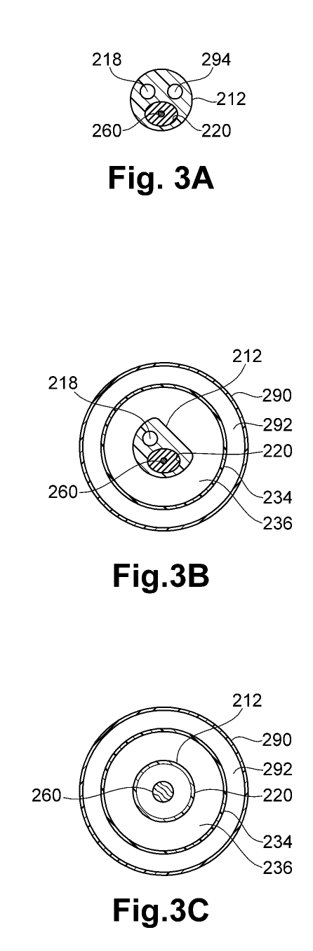

FIGS. 3, 3A, 3B, and 3C illustrate a third exemplary catheter 210. The catheter 210 illustrated in FIG. 3 is similar to the first exemplary catheter 10 illustrated in FIGS. 1 and 1A, except as detailed below. Thus, the catheter 210 includes an elongate main body 212 extending between proximal 214 and distal 216 ends. The main body 212 defines a first inflation lumen 218 and an optic lumen 220. The first inflation lumen 218 extends between an inflation port 222 and an opening 224 positioned on the distal end 216 of the main body 212. The optic lumen 220 extends from the proximal end 214 to the distal end 216 of the main body 212.

The catheter 210 includes a second balloon 290 that surrounds the first balloon 234. The second balloon 290 defines an infusion chamber 292 with the exterior surface of the main body 212 and the first balloon 234, and the first balloon 234 is disposed within the infusion chamber 292. The main body 212 also defines an infusion lumen 294 and a second opening 296 that are in fluid communication with the infusion chamber 292. Similar to the first balloon 234, the second balloon 290 has expanded and unexpanded configurations. The second balloon is moved between these configurations by way of movement of the first balloon 234 between its inflated and uninflated configurations. Thus, a user expands the second balloon 290 by inflating the first balloon 234. To move the second balloon 290 to the unexpanded configuration, vacuum pressure can be applied to the first inflation lumen 218 to remove fluid located within the first inflation chamber 236. FIGS. 3, 3B, and 3C illustrate the second balloon 290 in the expanded configuration.

The second balloon 290 advantageously has memory imparted onto it by a heat treatment step comprising heating the second balloon while it is in the unexpanded configuration. This heat treatment and the resulting memory gives the second balloon a tendency to return to its unexpanded configuration, which allows it to do so when the first balloon is moved from its inflated configuration to its deflated configuration.

The second balloon 290 includes one or more regions 298 that include one or more pores 300, each of which extend through the material of the second balloon 290 and permit fluid to pass through, preferably with the application of pressure. Suitable diameters for each of the one or more pores 300 include diameters in the range from about 0.01 mm to about 0.25 mm. Additional exemplary diameters for each of the one or more pores 300 include diameters in the range from about 0.025 to about 0.15. Further exemplary diameters for each of the one or more pores 300 include diameters in the range from about 0.05 mm to about 0.1 mm. Alternatively, when at least two or more pores 300 are provided, at least two of the pores 300 have different diameters.

The regions 298 can be positioned and/or arranged on the balloon 290 in any suitable configuration. For example, as illustrated in FIG. 3, an intermittent “striping” of the second balloon 290 with circumferentially-extending regions 298 can be positioned around the second balloon 290. Other contemplated configurations that are expected to provide specific utility include a single circumferentially-extending region positioned substantially on the axial midpoint of the second balloon 290, and a single circumferentially-extending region positioned substantially on the distal half, third, two-thirds, fourth, or three-fourths of the second balloon 290.

While the second balloon 290 has been described and illustrated as including regions 298 that include one or more pores 300, the second balloon 290 alternatively could be a solid piece of material that lacks regions 298 and pores 300. This configuration advantageously provides for two different inflated configurations of the catheter 210. For example, the first balloon 234 can be expanded to an inflated configuration (e.g., a diameter of about 5 mm) and the second balloon 290 can be left uninflated. If necessary or desired, the second balloon 290 can then be expanded to an inflated configuration to provide a greater diameter (e.g., a diameter of about 7 mm) than that provided if only the first balloon 234 was expanded to an inflated configuration.

In the illustrated embodiment, the fiber optic 260 is masked in a way that creates unmasked portions 302 that align orthogonally with the regions 298 on the second balloon 290, with respect to the lengthwise axis of the catheter 210 and/or main body 212. In contrast with the first 10 and second 110 exemplary catheters, which include masking structure that allows radially-directed light being emitted from the respective fiber optic to extend along the entire working length of the respective balloon, this structural arrangement of the regions 298 and the unmasked portions 302 allows radially-directed light being emitted from the fiber optic 260 to align with the regions 298 of the second balloon 290. This structural arrangement enables a user of the catheter 210 to transcutaneously identify the portion of a body vessel and/or cavity that lies adjacent to the regions 298 of the second balloon 290 that include pores 300. Alternatively, or in addition to transcutaneous identification, the user can identify the portion of the body vessel and/or cavity that lies adjacent the regions 298 of the second balloon 290 using direct visualization with a scope or other suitable device prior to providing treatment. If fluid is delivered through the infusion lumen 294 with sufficient pressure such that the fluid is expelled through the pores 300, the user can transcutaneously identify the portion or portions of a body vessel or cavity that will be contacted by the fluid when it exits the pores 300. If a drug or other bioactive substance is included in the fluid, the user will be able to transcutaneously or directly visualize and identify the portion or portions of a body vessel or cavity that will be exposed to the drug or other bioactive, before forcing the drug or bioactive through the pores.

As an alternative arrangement, one or both of the balloons 234, 290 could be masked along the entire axial length thereof except for in regions that contain or that are aligned orthogonally with the pore-containing regions 298 of the second balloon 290 with respect to the lengthwise axis of the catheter 210 and/or main body 212. Also alternatively, one or both balloons 234, 290 could be masked in the opposite arrangement. That is, one or both balloons 234, 290 could be masked in regions that contain or that are aligned orthogonally with the pore-containing regions 298 of the second balloon 290, leaving the other portions of the balloon(s) unmasked. In this embodiment, the structural adaptations of the fiber optic 260 that produce the radially-emitting light could be omitted.

Catheters can include additional features and/or components. For example, a catheter according to an embodiment can include a camera disposed on, in, or near the distal end of the main body, adjacent the balloon or balloons, or at any other suitable location on the catheter. If included, the camera is advantageously configured to transmit images to a display for a user to view during use of the catheter. Inclusion of a camera, while optional, is considered advantageous at least because it can facilitate direct visualization of radially-directed light, eliminating or reducing the need to rely on transcutaneous visualization of radially-directed light.

If included, any camera suitable for inclusion in a medical device such as a catheter can be used. A skilled artisan will be able to select an appropriate camera and associated components, if necessary, according to a variety of considerations, including the nature of the body vessel within which the catheter is intended to be used, the desired quality and/or size of the images to be transmitted by the camera, and other considerations. The camera can include any device suitable for capturing and transmitting an image via wire and/or wirelessly to a display. For example, if the camera is wired to a display, the wire connecting the camera to the display can have a distal end connected to the camera, a length disposed through the catheter, such as through the optic lumen or through an additional lumen defined by the main body of the catheter, and a proximal end connected to a display device, memory device, and/or another suitable apparatus adapted to receive, store, and/or display images transmitted by the camera through the wire. If a wireless camera is used, only the camera and a suitable apparatus for receiving, storing, and/or displaying the images transmitted wirelessly by the camera need be included.

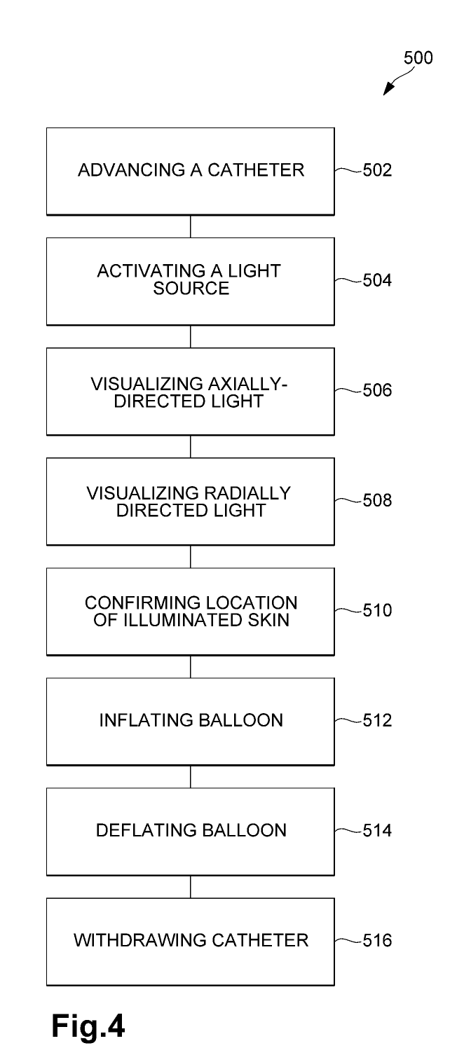

FIG. 4 is a flowchart representation of a method 500 of transcutaneously identifying and treating bodily passages. In a first step 502, a user advances a catheter into a bodily passage of a patient using conventional interventional techniques, such as by passing the catheter through a guiding catheter to direct the catheter to a point of treatment. Alternatively, the user advances the catheter into the body vessel independent of any other device. The catheter comprises an elongate main body having a proximal end and a distal end and defines an inflation lumen and an optic lumen. A balloon is attached to the distal end of the main body and adapted to move between deflated and inflated configurations, and has a proximal portion, a distal portion, and an intermediate portion disposed between the proximal and distal portions. The intermediate portion of the balloon defines a maximum diameter of the balloon when the balloon is in the inflated configuration. The balloon is in fluid communication with the inflation lumen via an opening defined by the main body of the catheter. The catheter also comprises a light source and a fiber optic disposed in the optic lumen. The fiber optic is operatively connected to the light source and is adapted to emit light radially along the intermediate portion of the balloon and distally from the distal end of the main body, such as by including the proximal 62 and distal 64 masked portions as described herein in connection with the first exemplary catheter 10. Alternatively, the main body, optic lumen, balloon, and/or fiber optic can be adapted to allow for only axially-directed light to emit from the distal end of the main body.

In another step 504, the user activates the light source, causing radially-directed and/or axially light to emit from the fiber optic along the intermediate portion of the balloon and/or axially-directed light to emit from the distal end of the catheter.

In another step 506, the user visualizes radially- and/or axially-directed light through the skin of the patient and advances the distal end of the catheter into the portion of the bodily passage intended to be treated by the catheter until the user determines that the intensity of the radially- and/or axially-directed light is indicative of positioning of the distal end of the catheter in the portion of the bodily passage intended to be treated.

In another step 508, the user visualizes radially- and/or axially-directed light through the wall of the balloon either through the skin of the patient and/or using direct visualization with a scope or other suitable device.

If the catheter is adapted to emit radially-directed light, in another step 510, the user confirms that the radially-directed light is illuminating a portion of the skin of the patient that is located superficially to the portion of the bodily passage intended to be treated by the catheter.

In another step 512, the user inflates the balloon of the catheter. This step can be used to perform dilation of the bodily passage. For example, in a balloon sinuplasty procedure, this step can be used to perform dilatation of a blocked sinus passage. If the bodily passage is a sinus cavity, this step can be conducted until the inflation of the balloon causes breakage of bone surrounding, forming, or partially forming the sinus cavity, if desired.

In another step 514, the user deflates the balloon of the catheter.

In another step 516, the user withdraws the catheter from the bodily passage.

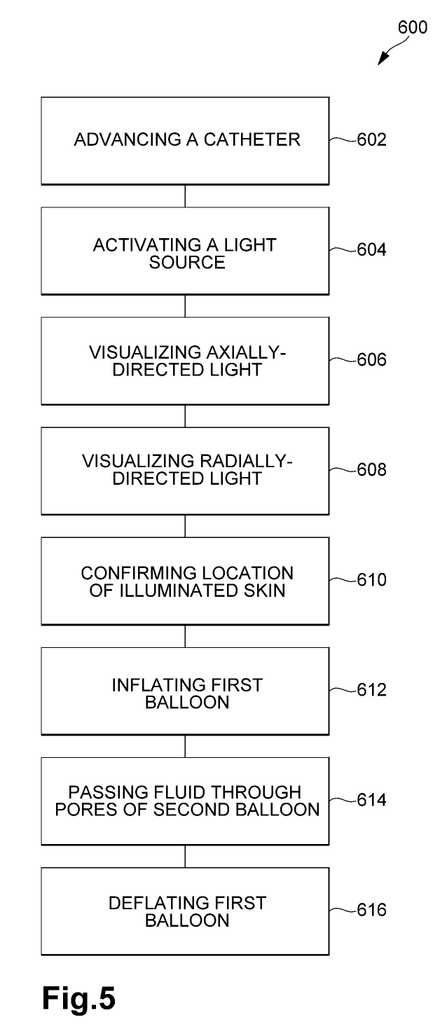

FIG. 5 is a flowchart representation of a method 600 of transcutaneously identifying and treating bodily passages. In a first step 602, a user advances a catheter into a bodily passage of a patient using conventional interventional techniques. The catheter comprises an elongate main body having a proximal end and a distal end and defines an inflation lumen, an infusion lumen, and an optic lumen. A first balloon is attached to the distal end of the main body and adapted to move between deflated and inflated configurations, and has a proximal portion, a distal portion, and an intermediate portion disposed between the proximal and distal portions. The intermediate portion of the balloon defines a maximum diameter of the balloon when the balloon is in the inflated configuration. The first balloon is in fluid communication with the inflation lumen via a first opening defined by the main body of the catheter. A second balloon is attached to the distal end of the main body and is disposed around the first balloon such that the first balloon is contained entirely within the infusion chamber defined by the second balloon. The second balloon is adapted to move between expanded and unexpanded configurations as the first balloon moves between inflated and uninflated configurations. The second balloon is in fluid communication with the infusion lumen via a second opening defined by the main body of the catheter. The second balloon includes one or more regions that include pores. The fiber optic is masked in a way that creates unmasked portions that align orthogonally with the regions on the second balloon that define the pores, with respect to the lengthwise axis of the catheter. The catheter also comprises a light source and a fiber optic disposed in the optic lumen. The fiber optic is operatively connected to the light source and is adapted to emit light radially along the intermediate portion of the balloon only in the regions on the second balloon that define the pores, with respect to the lengthwise axis of the catheter, and distally from the distal end of the main body.

In another step 604, the user activates the light source, causing radially-directed light to emit from the fiber optic along the intermediate portion of the balloon only in the regions on the second balloon that define the pores, and axially-directed light to emit from the distal end of the catheter. Alternatively, the user activates the light source causing only axially-directed light to emit from the fiber optic and the distal end of the catheter.

In another step 606, the user visualizes the axially-directed light through the skin of the patient and advances the distal end of the catheter into the portion of the bodily passage intended to be treated by the catheter until the user determines that the intensity of the axially-directed light is indicative of positioning of the distal end of the catheter in the portion of the bodily passage intended to be treated.

In another step 608, the user visualizes the radially-directed light through the skin of the patient or directly using a scope or other suitable device.

In another step 610, the user confirms that the radially-directed light is aligned with the tissue intended to be treated by the catheter.

In another step 612, the user inflates the first balloon of the catheter, which also expands the second balloon. If dilatation of the bodily passage is desired, this step can be used to perform the dilatation. For example, the first balloon can be inflated until a desired degree of dilatation is achieved, preferably before initiating the step 614. As described above, if the bodily passage is a sinus cavity and dilatation is desired, this step can be conducted until the inflation of the first balloon causes breakage of bone surrounding, forming, or partially forming the sinus cavity, if desired. If dilatation of the bodily passage is not desired, this step can be conducted until the first balloon is expanded to a point that causes a sufficient and/or desired degree of expansion of the second balloon, preferably before initiating the step 614.

In another step 614, the user passes fluid into the infusion lumen with sufficient pressure to force fluid through the pores defined by the second balloon.

In another step 616, the user deflates the first balloon of the catheter, which results in the second balloon returning to its unexpanded configuration.

In another step (not referenced in the Figure), the user withdraws the catheter from the bodily passage.

Other methods of treatment are also contemplated. For example, the dual-balloon catheters disclosed herein can be used for dilatation and/or drug infusion in suitable bodily passages with or without the use of the fiber optic and the axially- and radially-directed light it produces. In addition, a single balloon structure, such as that illustrated in FIG. 1, can be modified to include the pores of the outer balloon of the dual-balloon structure and used as a convenient drug infusion catheter for bodily passages, such as the sinus cavities and portions of the airway. In these embodiments and methods, catheters can be used for dilatation and/or drug infusion in suitable bodily passages with or without the use of the fiber optic and the axially- and radially-directed light it produces. Furthermore, the dual-balloon catheters disclosed herein can be modified to include an outer balloon which omits the regions containing the pores.

It is noted that the inventive methods can be used in any suitable bodily passage in any suitable animal. For example, the methods can be used in the treatment of human beings and other animals, and can be used in the treatment of sinus cavities, airway passages, and any other bodily passage for which treatment is desired. While the use of the transcutaneous identification features and steps are considered particularly advantageous for inclusion in methods used in relatively superficial bodily passages, such as sinus cavities, the inventors do not consider the apparatuses or methods limited to use in such passages.

The foregoing detailed description provides exemplary embodiments of the invention and includes the best mode for practicing the invention. The description and illustration of embodiments is intended only to provide examples of the invention, and not to limit the scope of the invention, or its protection, in any manner.

We claim:

1. A catheter adapted to be used with a light source for treatment and transcutaneous identification of a sinus passage, said catheter having a lengthwise axis and comprising:
an elongate main body having a proximal end and a distal end, the main body defining an inflation lumen and an optic lumen;
a balloon attached to the distal end of the main body and adapted to move between deflated and inflated configurations, the balloon having a first proximal portion, a first distal portion, and a first intermediate portion disposed between the first proximal and first distal portions, the first intermediate portion defining a maximum diameter of the balloon when the balloon is in the inflated configuration; and
a fiber optic disposed in the optic lumen and adapted to be operatively connected to said light source, the fiber optic having a second proximal portion, a second distal portion, and a second intermediate portion disposed between the second proximal and second distal portion, the fiber optic adapted to emit light distally from the distal end of the main body.

2. The catheter of claim 1, wherein the second proximal portion and second distal portion are adapted to substantially prevent light from escaping from the fiber optic.

3. The catheter of claim 2, wherein the second intermediate portion is adapted to emit light radially from the fiber optic.

4. The catheter of claim 3, wherein the first intermediate portion has a first axial length and the second intermediate portion has a second axial length; and
wherein the first axial length is substantially equal to the second axial length.

5. The catheter of claim 4, wherein the fiber optic is fixed in position relative to the elongate main body.

6. The catheter of claim 5, wherein the first intermediate portion has a second proximal end and a second distal end;
wherein the second intermediate portion has a third proximal end and a third distal end; and
wherein the second proximal end and the third proximal end are aligned on a first orthogonal axis that intersects the lengthwise axis of the catheter, and the second distal end and the third distal end are aligned on a second orthogonal axis that intersects the lengthwise axis of the catheter.

7. The catheter of claim 1, wherein the first intermediate portion is adapted to allow light emitted from the second intermediate portion to pass through the balloon to an external environment.

8. The catheter of claim 7, wherein the first proximal portion and first distal portion of the balloon are opaque.

9. The catheter of claim 1, further comprising a soft distal tip attached to the second distal portion of the fiber optic.

10. The catheter of claim 1, wherein the fiber optic is fixed in position relative to the elongate main body.

11. The catheter of claim 1, wherein a second balloon is attached to the distal end of the main body and is disposed over the balloon of the catheter.

12. A catheter adapted to be used with a light source for treatment and transcutaneous identification of a sinus passage, said catheter having a lengthwise axis and comprising:
an elongate main body having a proximal end and a distal end, the main body defining an inflation lumen and an optic lumen;
a balloon attached to the distal end of the main body and adapted to move between deflated and inflated configurations, the balloon having an opaque proximal portion, an opaque distal portion, and a transparent intermediate portion disposed between the proximal and distal portions, the transparent intermediate portion defining a maximum diameter of the balloon when the balloon is in the inflated configuration; and
a fiber optic disposed in the optic lumen and adapted to be operatively connected to said light source, the fiber optic having a second proximal portion, a second distal portion, and a second intermediate portion disposed between the second proximal and second distal portion, the fiber optic adapted to emit light radially along the transparent intermediate portion of the balloon and distally from the distal end of the main body.

13. The catheter of claim 12, wherein the opaque intermediate portion has a first axial length and the second intermediate portion has a second axial length; and
wherein the first axial length is substantially equal to the second axial length.

14. The catheter of claim 13, wherein the fiber optic is fixed in position relative to the elongate main body.

15. The catheter of claim 14, wherein the opaque intermediate portion has a second proximal end and a second distal end;
wherein the second intermediate portion has a third proximal end and a third distal end; and
wherein the second proximal end and the third proximal end are aligned on a first orthogonal axis that intersects the lengthwise axis of the catheter, and the second distal end and the third distal end are aligned on a second orthogonal axis that intersects the lengthwise axis of the catheter.

16. The catheter of claim 12, wherein a second balloon is attached to the distal end of the main body and is disposed over the balloon of the catheter.

17. A catheter adapted to be used with a light source for treatment and transcutaneous identification of a sinus passage, said catheter having a lengthwise axis and comprising:
an elongate main body having a proximal end and a distal end, the main body defining an inflation lumen and an optic lumen;
a balloon attached to the distal end of the main body and adapted to move between deflated and inflated configurations, the balloon having an opaque proximal portion, an opaque distal portion, and a transparent intermediate portion disposed between the proximal and distal portions, the intermediate portion defining a maximum diameter of the balloon when the balloon is in the inflated configuration; and
a fiber optic disposed in the optic lumen and adapted to be operatively connected to said light source, the fiber optic having a second proximal portion, a second distal portion, and a second intermediate portion disposed between the second proximal and second distal portion, the fiber optic adapted to emit light radially along the transparent intermediate portion of the balloon and distally from the distal end of the main body;
wherein the fiber optic is fixed in position relative to the elongate main body.

18. The catheter of claim 17, wherein the opaque intermediate portion has a first axial length and the second intermediate portion has a second axial length; and
wherein the first axial length is substantially equal to the second axial length.

19. The catheter of claim 18, wherein the opaque intermediate portion has a second proximal end and a second distal end;
wherein the second intermediate portion has a third proximal end and a third distal end; and
wherein the second proximal end and the third proximal end are aligned on a first orthogonal axis that intersects the lengthwise axis of the catheter, and the second distal end and the third distal end are aligned on a second orthogonal axis that intersects the lengthwise axis of the catheter.

20. The catheter of claim 1, wherein a second balloon is attached to the distal end of the main body and is disposed over the balloon of the catheter.
